(19) United States
(12) Patent Application Publication
(10) Pub. No.: US 2020/0108112 A1
(43) Pub. Date: Apr. 9, 2020

(54) METHOD FOR PREPARING FENUGREEK EXTRACT AND PHARMACEUTICAL COMPOSITION COMPRISING FENUGREEK EXTRACT

(71) Applicant: PRINCE PHARMACEUTICAL CO., LTD., New Taipei City (TW)

(72) Inventor: Sih-Yu SHEN, New Taipei City (TW)

(73) Assignee: PRINCE PHARMACEUTICAL CO., LTD., New Taipei City (TW)

(21) Appl. No.: 16/703,418

(22) Filed: Dec. 4, 2019

Related U.S. Application Data
(62) Division of application No. 15/882,001, filed on Jan. 29, 2018.

(30) Foreign Application Priority Data
May 18, 2017 (TW) 106116461

(51) Int. Cl. A61K 36/48; A61P 1/16; A61K 36/25; A23L 33/105; A61K 36/28; A61K 36/899; A61K 36/258

(52) U.S. Cl. CPC A61K 36/48; A61P 1/16; A61K 36/25; A61K 2236/37; A61K 36/28; A61K 36/899; A61K 36/258; A23L 33/105

(57) ABSTRACT

Provided is a method for preparing a fenugreek extract, including the steps of: (1) preparing a fenugreek plant tissue, soaking the fenugreek plant in water from 0.5 hour to 5 hours and between 25° C. and 100° C. for extraction, and (2) filtering the extracted fenugreek plant tissue to obtain the fenugreek extract. Also provided is an active substance that comprises the fenugreek extract obtained by the method. Also provided is a pharmaceutical composition for preventing or treating nonalcoholic fatty liver disease, which comprises the active substance and a pharmaceutically acceptable carrier.

### CROSS REFERENCE

This application is a divisional application of United States patent application filed on Jan. 29, 2018 and having application Ser. No. 15/882,001, the entire contents of which are hereby incorporated herein by reference.

This application claims priority under 35 U.S.C. § 119(a) to Taiwan Patent Application No. 106116461, filed on May 18, 2017, the content of which is hereby incorporated by reference in its entirety.

### BACKGROUND OF THE INVENTION

### 1. Field of the Invention

The present invention relates to a method, especially a method for preparing a fenugreek extract by water extraction. The present invention also relates to an active substance which comprises the fenugreek extract obtained by the method and red yeast rice extract for preventing or treating nonalcoholic fatty liver disease. The present invention further relates to a pharmaceutical composition, and particularly, the pharmaceutical composition comprises the fenugreek extract obtained by the method, red yeast rice extract, rice bran, artichoke, taurine, ginseng extract or any combination thereof.

### 2. Description of the Prior Arts

Fatty liver is the accumulation of triglycerides and other fats in the liver cells. When the diet intake of fat is more than the body needs, adipose tissues will gradually accumulate in the liver. If the fat accounts for more than 5% of the liver, the symptom is called fatty liver. Fat accumulation in the liver causes continuous damage to the liver, leading to hepatitis and scarring of the liver. Non-alcoholic fatty liver disease (NAFLD) is a progressive, complicated disease initiated by fat accumulation in the liver, while the fat accumulation is not caused by excessive alcohol. With excessive fat accumulation and degeneration, patients with NAFLD may develop nonalcoholic steatohepatitis (NASH) including hepatitis, necrosis, and fibrosis. Steatohepatitis means continuous damage of the fatty liver disease with inflammation, which is similar to alcoholic steatohepatitis but occurs in nondrinkers or people who drink small amounts of alcohol. NASH is different to the fat accumulation in liver, as the physical condition of people with the latter can still be good, while 20% of the adults who suffer from NASH would develop cirrhosis, 11% would die due to fatal liver disease, and many cases of chronic liver failure need liver transplantation.

Fenugreek (Trigonella foenum-graecum), an annual plant used as an herb, spice, and vegetable, has been demonstrated to have anti-oxidant, anti-hyperlipidemic, and anti-hyperglycemic activities, and relieves sinus and asthma symptoms by mucus reduction. Most of the prior arts obtain plant extracts using organic solvents such as methanol, ethanol (alcohol), acetone, ethyl acetate and so on. However, the organic solvent is easy to remain in the extract and is not easily separated. As people now place more emphasis on food safety, the plant extraction method needs to be further improved.

### SUMMARY OF THE INVENTION

To overcome the defects of the prior arts, the object of the present invention is to provide a method for preparing a fenugreek extract, which has efficacy on preventing or treating nonalcoholic fatty liver disease.

To achieve the above object, the present invention provides a method for preparing a fenugreek extract, comprising the steps of:

(1) preparing a fenugreek plant tissue, soaking the fenugreek plant tissue in water for 0.5 hour to 5 hours and at a temperature between 25° C. and 100° C. for extraction, to obtain an extracted fenugreek plant tissue; and

(2) filtering the extracted fenugreek plant tissue to obtain the fenugreek extract.

Preferably, the fenugreek plant tissue described in the foresaid step (1) is fenugreek seed or whole-plant fenugreek.

Preferably, in the foresaid step (1), the water is at a weight ratio of 1 to 20 times of the fenugreek plant tissue.

Preferably, in the foresaid step (1), the extraction period is between 0.5 hours and 3 hours, and the temperature of the extraction is between 50° C. and 95° C.

The present invention further provides a fenugreek extract obtained by the method as described above.

The present invention further provides an active substance for preventing or treating nonalcoholic fatty liver disease. The active substance comprises the fenugreek extract described above.

Preferably, the active substance further comprises red yeast rice extract, rice bran extract, artichoke extract, taurine, ginseng extract or any combination thereof.

More preferably, the active substance comprises 12 to 16 parts by weight fenugreek extract, 3 to 14 parts by weight red yeast rice extract, 0.5 to 5 parts by weight rice bran extract, 0.5 to 3 parts by weight artichoke extract, 0.8 to 25 parts by weight taurine, and 0.1 to 10 parts by weight ginseng extract.

The present invention further provides a pharmaceutical composition for preventing or treating nonalcoholic fatty liver disease, wherein the pharmaceutical composition comprises the active substance described above and a pharmaceutically acceptable carrier.

The present invention further provides a method for preventing or treating nonalcoholic fatty liver disease with the pharmaceutical composition described above, which is administering to a subject a therapeutically effective amount of the pharmaceutical composition to achieve the effect of preventing or treating nonalcoholic fatty liver disease.

The active substance of the present invention can be adopted with a pharmaceutically acceptable carrier through a well-known technique to prepare a dosage form suitable for use in the pharmaceutical composition of the present invention. The pharmaceutical composition described herein may comprise well-known excipient in the art, including, but not limited to, plasticizer, filler, lubricant, diluent, binder, disintegrant, solvent, interfacial active agent, preservative, sweetener, antioxidant and viscosity agent. Preferably, the plasticizer includes, but is not limited to, corn flour. Preferably, the lubricant includes, but is not limited to, silicon dioxide (SiO₂), and magnesium stearate (MAG). Preferably, the diluent includes, but is not limited to, maltodextrin)(Fibersol-2®). Preferably, the antioxidant includes, but is not limited to, vitamin E.

The pharmaceutical composition of the present invention may be presented in a variety of forms including, but not limited to, solid pharmaceutical form. The solid pharmaceutical form includes, but is not limited to, capsule, troche, pill, powder, liposome and suppository. The preferred form depends on the intended mode of administration and the therapeutic application; preferably, the dosage form of the pharmaceutical composition of the present invention is oral administration.

According to the present invention, the term “therapeutically effective amount” as used herein, refers to an amount effective in preventing and treating nonalcoholic fatty liver disease in terms of dosage and for the desired time; as exemplified by the present invention, effective prevention of nonalcoholic fatty liver disease can be measured by lipid droplets formation assay, liver slide staining, total cholesterol, the levels of serum glutamate oxaloacetic transaminase (sGOT) and serum glutamic-pyruvic transaminase (sGPT) of blood analysis.

Preferably, the active substance comprises the fenugreek extract, red yeast rice extract, rice bran extract, artichoke extract, taurine, and ginseng extract, orally administered to the subject between 0.0004 g per kg per day and 0.81 g per kg per day. The effective amount of the active substance administered to the subject is calculated based on the initial experimental method of the US Food and Drug Administration published in 2005: estimating the maximum safe starting dose in initial clinical trials for therapeutics in adult healthy volunteers.

The fenugreek extract extracted by the water extraction preparation example method of the present invention has the advantage that the fenugreek extract extracted by the water extraction preparation example method of the present invention has better efficacy in preventing or treating nonalcoholic fatty liver than the fenugreek extract extracted with an organic solvent. The fenugreek extract extracted by the water extraction preparation method has better efficacy in inhibiting fatty liver than the fenugreek extract extracted with an organic solvent at the same dose. In addition, when the fenugreek extract is used in combination with red yeast rice extract, rice bran extract, artichoke extract, taurine, and ginseng extract, the effect of preventing or treating nonalcoholic fatty liver is better than using the fenugreek extract alone.

Other objectives, advantages and novel features of the invention will become more apparent from the following detailed description when taken in conjunction with the accompanying drawings.

### BRIEF DESCRIPTION OF THE DRAWINGS

FIG. 1 is an index histogram of lipid droplets formation in Group A (fenugreek water extract obtained by preparation example 1) of the present invention; each group is duplicate, mean±standard deviation (SD), Student's t test, * for p<0.05, ** for p<0.01, *** for p<0.001, and MTS as an analytical method for cell viability.

FIG. 2 is an index histogram of lipid droplets formation in Group B (fenugreek alcohol extract obtained by preparation example 2) of the present invention; each group is duplicate, mean±SD, Student's t test, * for p<0.05, ** for p<0.01, and *** for p<0.001.

FIG. 3 is an index histogram of lipid droplets formation in Group C (fenugreek water extract obtained by preparation example 1) of the present invention; each group is duplicate, mean±SD, Student's t test, * for p<0.05, ** for p<0.01, and *** for p<0.001.

FIG. 4 is an index histogram of lipid droplets formation in Group D (fenugreek alcohol extract obtained by preparation example 2) of the present invention; each group is duplicate, mean±SD, Student's t test, * for p<0.05, and *** for p<0.001.

FIG. 5 is a graph of the H&E staining of liver tissue slides of the control group, high fat diet group and YE2 group (fenugreek water extract obtained by preparation example 1) of the present invention.

FIG. 6 is a histogram of the macrovesicular steatosis score of the control group, the high fat diet group and the YE2 group, mean±SD, one-way ANOVA; * represents a comparison with the control group p<0.05; † represents a comparison with the high fat diet group p<0.05.

FIG. 7 is a histogram of the microvesicular steatosis score of the control group, the high fat diet group and the YE2 group, mean±SD, one-way ANOVA; * represents a comparison with the control group p<0.05; † represents a comparison with the high fat diet group p<0.05.

FIG. 8 is a histogram of the ballooning degeneration score of the control group, the high fat diet group and the YE2 group, mean±SD, one-way ANOVA; * represents a comparison with the control group p<0.05; † represents a comparison with the high fat diet group p<0.05.

FIG. 9 is a histogram of the total cholesterol content of the control group, the high fat diet group and the YE2 group, mean±SD, one-way ANOVA; * represents a comparison with the control group p<0.05; † represents a comparison with the high fat diet group p<0.05; mg/L.

FIG. 10 is a histogram of the serum GOT level of the control group, the high fat diet group and the YE2 group, mean±SD, one-way ANOVA; * represents a comparison with the control group p<0.05; † represents a comparison with the high fat diet group p<0.05; U/L.

FIG. 11 is a histogram of the serum GPT level of the control group, the high fat diet group and the YE2 group, mean±SD, one-way ANOVA; * represents a comparison with the control group p<0.05; † represents a comparison with the high fat diet group p<0.05; U/L.

FIG. 12 is an index histogram of lipid droplets formation of the fenugreek water extract formula of the present invention; each group is four-repeat, mean±SD, Student's t test, *** for p<0.001.

FIG. 13 is a histogram of cell viability of HepG2 cells; each group is four-repeat, mean±SD, Student's t test, *** for p<0.001.

FIG. 14 is a histogram of the serum triglyceride levels; each group is ten-repeat, mean±SD; values with different superscripts are significantly different at p<0.05 by one-way ANOVA with Duncan's multiple range test.

FIG. 15 is a histogram of the serum cholesterol levels; each group is ten-repeat, mean±SD; values with different superscripts are significantly different at p<0.05 by one-way ANOVA with Duncan's multiple range test.

FIG. 16 is a histogram of the serum non-esterified fatty acid (referred to as serum NEFA) levels; each group is ten-repeat, mean±SD; values with different superscripts are significantly different at p<0.05 by one-way ANOVA with Duncan's multiple range test.

FIG. 17 is a histogram of the serum ALT levels; each group is ten-repeat, mean±SD; values with different superscripts are significantly different at p<0.05 by one-way ANOVA with Duncan's multiple range test.

FIG. 18 is a histogram of the serum AST levels; each group is ten-repeat, mean±SD; values with different superscripts are significantly different at p<0.05 by one-way ANOVA with Duncan's multiple range test.

FIG. 19 is a graph of the H&E staining of liver tissue slides of each group.

FIG. 20 is a histogram of the total liver cholesterol levels of each group; each group is ten-repeat, mean±SD; values with different superscripts are significantly different at p<0.05 by one-way ANOVA with Duncan's multiple range test.

FIG. 21 is a histogram of the total liver triglyceride levels of each group; each group is ten-repeat, mean±SD; values with different superscripts are significantly different at p<0.05 by one-way ANOVA with Duncan's multiple range test.

FIG. 22 is a histogram of the total liver non-esterified fatty acid (referred to as Total liver NEFA) levels of each group; each group is ten-repeat, mean±SD; values with different superscripts are significantly different at p<0.05 by one-way ANOVA with Duncan's multiple range test.

FIG. 23 is a high performance liquid chromatography (referred to as HPLC) profile of the fenugreek water extract of the present invention for total saponin at OD 203 nm.

FIG. 24 is a HPLC profile of the fenugreek whole plant extract of the present invention for total saponin at OD 203 nm.

### DETAILED DESCRIPTION OF THE PREFERRED EMBODIMENTS

### Preparation Example 1: Water Extraction of Fenugreek

The fresh or dried fenugreek seed was extracted with water, wherein the water was at a weight ratio of 1 to 20 times, preferably 5 to 10 times the weight of the fenugreek seed. The fenugreek seed was soaked in the water for a period from half hour to 5 hours and at a temperature between 25° C. to 100° C., preferably for a period from half hour to 3 hours and at a temperature between 50° C. to 95° C., and then filtered (solid-liquid separation, such as by porosity, weight or density, carried out by any known physical filtration method) to obtain the fenugreek water extract (referred to as YE2). The fenugreek water extract could be concentrated or dried by any known food processing method.

### Preparation Example 2: Organic Solvent Extraction of Fenugreek

The fresh or dried fenugreek seed was extracted with 50% alcohol solvent, wherein the alcohol solvent was at a weight ratio of 1 to 20 times, preferably 5 to 10 times the weight of the fenugreek seed. The fenugreek seed was soaked in the alcohol solvent from half hour to 5 hours and between 25° C. to 100° C., preferably half hour to 3 hours and between 50° C. to 95° C., and then filtered to obtain the fenugreek alcohol extract (referred to as YE3). The fenugreek alcohol extract could be concentrated or dried by any known food processing method.

### Example 1 Cell Experiment: Simultaneous Administration and Stimulation, for Simulation of Prevention Mechanism

(1) HepG2 cells (liver hepatocellular carcinoma cells) were seeded at 2.5×10⁴ cells/well and incubated for 24 hours.

(2) The incubated cells were divided into the following groups:

Control group (CG) was added with cell culture medium and 1% dimethyl sulfoxide (DMSO) to treat the HepG2 cells for 6 hours.

Lipid droplets induced group (LDIG) was added with 1% DMSO and 500 mM lipid droplets induced reagent [oleic acid (OA), C18:1 and paltimic acid (PA), C16:0 were dissolved in the cell culture medium at a volume ratio of 2:1 (v/v)] to treat the HepG2 cells for 6 hours.

Positive control group (PCG) was added with 0.01 μg/mL phosphatidylcholine and 500 mM lipid droplets induced reagent to treat the HepG2 cells for 6 hours. Group A was added with the dried fenugreek water extract which was obtained by preparation example 1 and was dissolved in 1% DMSO to give a final concentration of 10⁻⁶ μm/mL, 10⁻⁵ μm/mL, 10⁻⁴ μm/mL, 10⁻³ μm/mL, 10⁻² μm/mL and 10⁻¹ μm/mL, respectively, simultaneously with 500 mM lipid droplets induced reagent to treat the HepG2 cells for 6 hours.

Group B was added with the dried fenugreek alcohol extract which was obtained by preparation example 2 and was dissolved in 1% DMSO to give a final concentration of 10⁻⁶ μm/mL, 10⁻⁵ μm/mL, 10⁻⁴ μm/mL, 10⁻³ μm/mL, 10⁻² μm/mL and 10⁻¹ μm/mL, respectively, simultaneously with 500 mM lipid droplets induced reagent to treat the HepG2 cells for 6 hours.

(3) The above groups were examined by cell viability assay (MTS assay) and oil red-O stain, and the oil red OD value/MTS OD value was used as the index of lipid droplets.

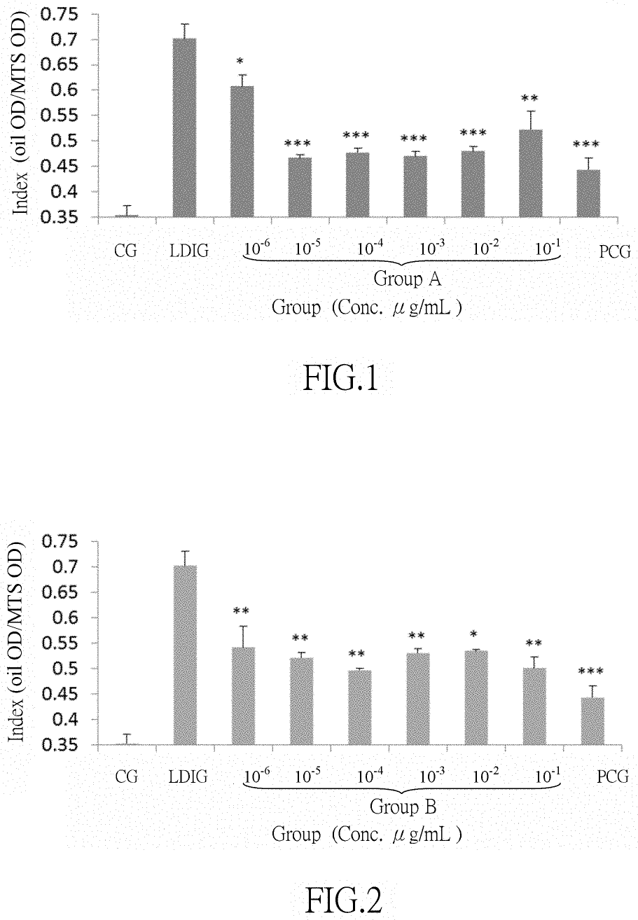

Referring to FIGS. 1 and 2, the fenugreek water extract and the fenugreek alcohol extract both dose-dependently inhibited lipid droplet formation in HepG2 cells. The half maximal inhibitory concentration (IC₅₀) of group A was 0.1 ng/mL, and the IC₅₀ of group B was 0.3 ng/mL. The fenugreek water extract at concentration 10⁻⁵ μg/mL in group A already has a great inhibitory effect.

### Example 2 Cell Experiment: Stimulation after Administration, for Simulation of Prevention Mechanism

(1) HepG2 cells were seeded at 2.5×10⁴ cells/well and incubated for 24 hours.

(2) The incubated cells were divided into the following groups:

Control group (CG) was added with 1% DMSO to treat the HepG2 cells for 16 hours, and then treated with cell culture medium for 16 hours.

Lipid droplets induced group (LDIG) was added with 1% DMSO to treat the HepG2 cells for 16 hours, and then treated with 500 nM lipid droplets induced reagent for 16 hours.

Positive control group (PCG) was added with 0.01 μg/mL phosphatidylcholine to treat the HepG2 cells for 16 hours, and then treated with 500 mM lipid droplets induced reagent for 16 hours.

Group C was added with the dried fenugreek water extract which was obtained by Preparation Example 1 and was dissolved in 1% DMSO to give a final concentration of 10⁻⁶ μm/mL, 10⁻⁵ μm/mL, 10⁻⁴ μm/mL, 10⁻³ μm/mL, 10⁻² μm/mL and 10⁻¹ μm/mL, respectively, and the HepG2 cells were treated for 16 hours, and then treated with 500 mM lipid droplets induced reagent for 16 hours.

Group D was added with the dried fenugreek alcohol extract which was obtained by Preparation Example 2 and was dissolved in 1% DMSO to give a final concentration of 10⁻⁶ μm/mL, 10⁻⁵ μm/mL, 10⁻⁴ μm/mL, 10⁻³ μm/mL, 10⁻² μm/mL and 10⁻¹ μm/mL, respectively, and the HepG2 cells were treated for 16 hours, and then treated with 500 mM lipid droplets induced reagent for 16 hours.

(3) The above groups were examined by cell viability assay (MTS assay) and oil red-O stain, and the oil red OD value/MTS OD value was used as the index of lipid droplets.

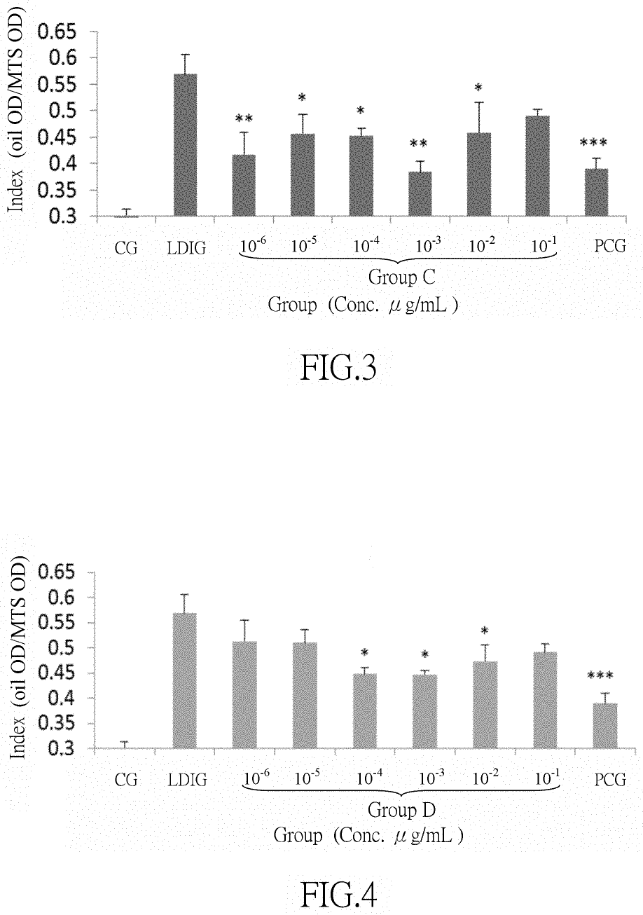

Referring to FIGS. 3 and 4, the IC₅₀ of group C was 0.3 ng/mL, and the IC₅₀ of group D was 0.9 ng/mL. The index value of the fenugreek water extract at concentration 10⁻³ μm/mL in group C was 0.3839±0.0203, having the greatest inhibitory effect on lipid droplets formation.

### Example 3: Animal Experiment

(1) 7 weeks old male C57BL/6J mice were chosen for the experiment.

(2) Divided into the following groups:

Control group (CG): 6 mice were fed with chow diet, including 4.8% kcal fat with 0% kcal cholesterol for 12 weeks.

High fat diet (HFD) group: 6 mice were fed with high fat diet, including 42% kcal fat with 0.2% kcal cholesterol for 12 weeks.

YE2 group: 6 mice were fed with high fat diet simultaneously with 0.108 g/kg/day the dried fenugreek water extract obtained by Preparation Example 1 for 12 weeks.

(3) The mice were sacrificed, and the liver tissue slides were stained by hematoxylin and eosin stain (H & E stain), according to the grading criteria for macrovesicular steatosis established by 2005 Kleiner et al.: fraction 0 was <5% (percentage of macrovesicular in the liver tissue), fraction 1 was 5% to 33%, fraction 2 was >33% to 66%, fraction 3 was >66%; for microvesicular steatosis: fraction 0 represented no occurrence of microvesicular steatosis, fraction 1 represented occurrence of microvesicular steatosis; and for ballooning degeneration: fraction 0 represented no ballooning degeneration, fraction 1 represented a small amount of ballooning degeneration, fraction 2 represented majority of ballooning degeneration. In addition, mice blood was extracted and analyzed for total cholesterol, sGOT and sGPT.

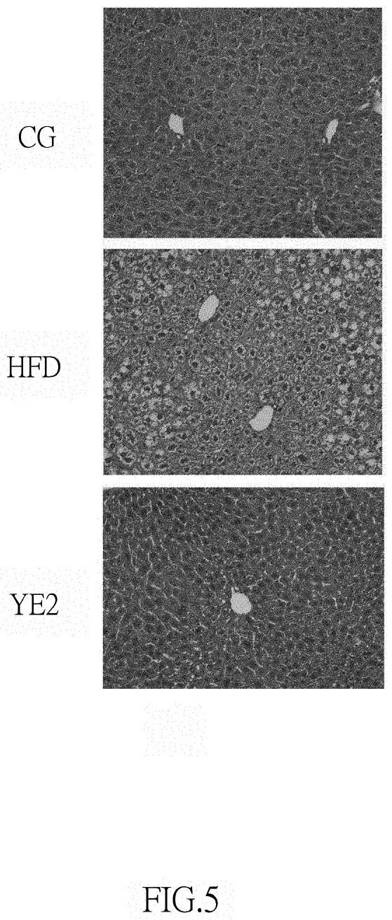

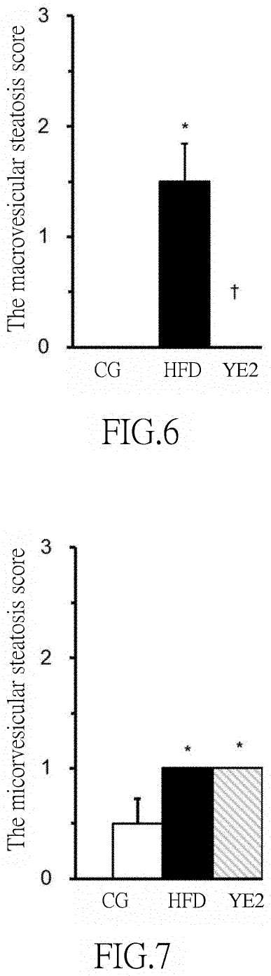

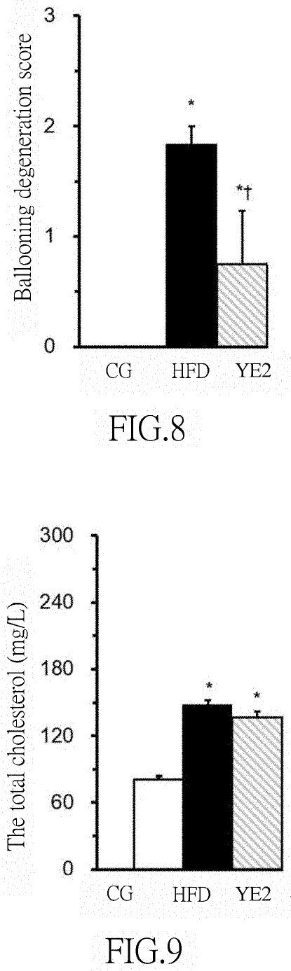

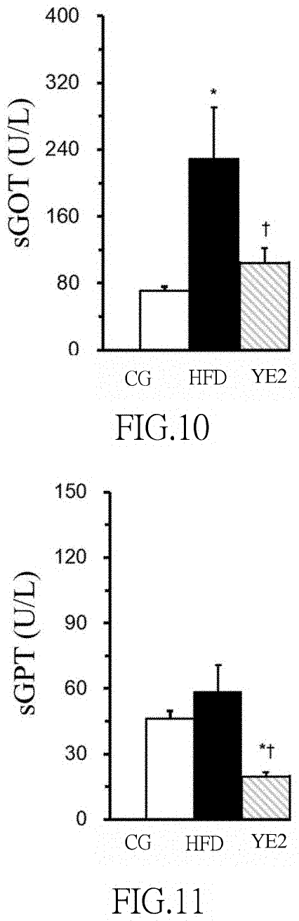

Referring to FIG. 5, the liver of the HFD group was filled with adipose, hollow with loose organization, while the YE2 group was more similar to the control group. Referring to FIG. 6, compared with the HFD group, the macrovesicular steatosis was significantly decreased in the YE2 group. Referring to FIG. 7, the level of microvesicular steatosis in the YE2 group was similar to the HFD group. Referring to FIG. 8, the YE2 group compared with the HFD group, the level of the ballooning degeneration was significantly decreased. Referring to FIG. 9, the total cholesterol content of the YE2 group was similar to that of the HFD group. Referring to FIG. 10, the YE2 group compared with the HFD group, the sGOT content was significantly decreased. Referring to FIG. 11, compared with the HFD group and the control group, the sGPT content was significantly decreased in the YE2 group.

### Example 4 Cell Experiment: Formula of Fenugreek Extract

(1) HepG2 cells were seeded at 2.5×10⁴ cells/well and incubated for 24 hours.

(2) The incubated cells were divided into the following groups:

Control group (CG) was added with 1% DMSO to treat the HepG2 cells for 16 hours, and then treated with cell culture medium for 16 hours.

Lipid droplets induced group (LDIG) was added with 1% DMSO to treat the HepG2 cells for 16 hours, and then treated with 500 nM lipid droplets induced reagent for 16 hours.

Dried fenugreek water extract which was obtained by Preparation Example 1, red yeast rice extract, rice bran extract, artichoke extract, taurine, and ginseng extract were mixed at different parts by weight into 8 experiment groups (EG) as shown in Table 1 below, wherein the latter five materials are commercially available. In addition, in a preferred embodiment, Vitamin E, SiO₂, MAG and Fibersol-2 ® may be additionally added as desired. Each experiment group was respectively added to treat the HepG2 cells for 16 hours, and then treated with lipid droplets induced reagent for 16 hours.

TABLE 1
Content of each group (parts by weight)

| | fenugreek | | | | | |
|---|---|---|---|---|---|---|
| | water | red yeast | rice bran | artichoke | | ginseng |
| | extract | rice extract | extract | extract | taurine | extract |
| 1 | — | — | — | — | — | — |
| 2 | 12~16 | 3~14 | 0.5~5 | 0.5~3 | 0.8~25 | 0.1~10 |
| 3 | 12~16 | — | — | — | — | — |
| 4 | — | 3~14 | — | — | — | — |
| 5 | — | — | 0.5~5 | — | — | — |
| 6 | — | — | — | 0.5~3 | — | — |
| 7 | — | — | — | — | 0.8~25 | — |
| 8 | — | — | — | — | — | 0.1~10 |

Note:
“—” means not added.

(3) The above groups were examined by MTS assay and oil red-O stain, and the oil red OD value/MTS OD value was used as the index of lipid droplets.

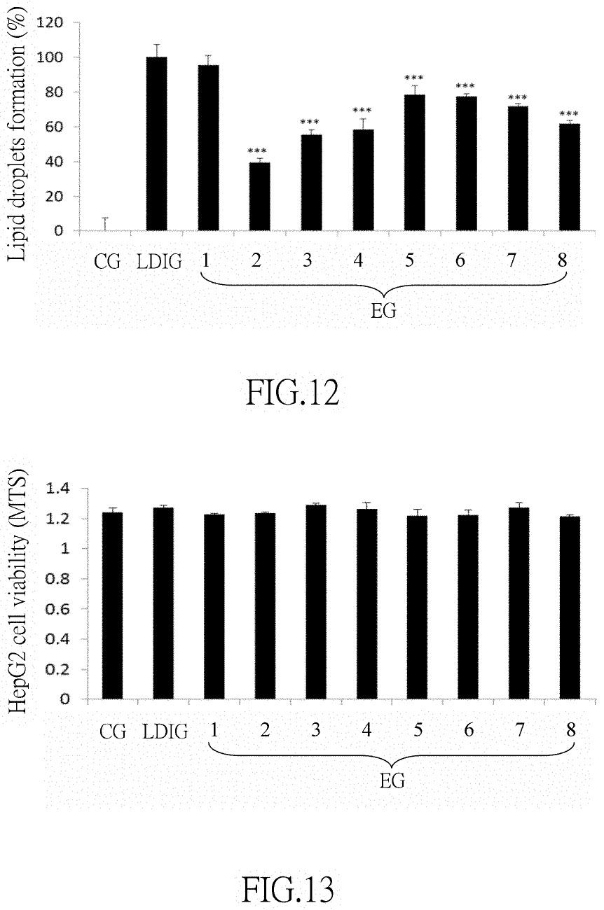

Referring to FIG. 12, the percentage of the lipid droplet formation of the lipid droplets induced group was 100%, the experiment group 1: 97%, the experiment group 2: 38.6%, the experiment group 3: 57%, the experiment group 4: 58.4%, the experiment group 5: 79.3%, the experiment group 6: 78.1%, the experiment group 7: 72.4%, and the experiment group 8: 60.1%. Lipid droplet formation was significantly decreased in the experiment groups 2 to 7, wherein the experiment group 2, which has all of the 6 materials, has the lowest percentage of lipid droplet formation.

Referring to FIG. 13, there was no significant change in cell viability OD ratio of the control group, the lipid droplets induced group, and the experiment groups 1 to 8, and thus the cell viability could be maintained in groups 2 to 8.

### Example 5 Animal Experiment—Formula of Fenugreek Extract

(1) 8 weeks old male C57BL/6J mice were chosen for the experiment.

(2) Divided into the following groups:

Control group (CG): 10 mice were given 0.2 mL ddH₂O daily and fed with a controlled diet after the start of the second week. The controlled diet was fed with general feed and tube-fed with ddH₂O, continuously until the 18th week.

High fat diet (HFD) group: 10 mice were given 0.2 mL ddH₂O daily and fed with a high fat diet after the start of the second week. The high fat diet was fed with 60% high fat feed and tube-fed with ddH₂O, continuously until the 18th week, wherein the 60% high fat feed was 20% kcal protein, 20% kcal carbohydrate and 60% kcal fat, the formula by E. A. Ulman, Ph. D., deployed in Research Diets, Inc., 8/26/98 With 3/11/99, product number D12492.

Low dose group (LDG): 10 mice were given 0.2 mL 1×(0.005˜1 g/kg/day) fenugreek water extract formula (same as the proportion of experiment group 2 in Example 4) daily and fed with the high fat diet after the start of the second week, continuously until the 18th week.

Medium dose group (MDG): 10 mice were given 0.2 mL 5×(0.025˜5 g/kg/day) fenugreek water extract formula (same as the proportion of experiment group 2 in Example 4, the medium dose group given 5 times the dose of the low dose group) daily and fed with the high fat diet after the start of the second week, continuously until the 18th week.

High dose group (HDG): 10 mice were given 0.2 mL 10×(0.05˜10 g/kg/day) fenugreek water extract formula (same as the proportion of experiment group 2 in Example 4, the high dose group given 10 times the dose of the low dose group) daily and fed with the high fat diet after the start of the second week, continuously until the 18th week.

(3) Blood samples were extracted for serum triglyceride, serum cholesterol, serum non-esterified fatty acid, serum ALT, and serum AST analysis at the 18th week. The mice were sacrificed at the 18th week to analyze the total cholesterol, total triglyceride and non-esterified fatty acid in the liver tissues, and the liver tissue slides were stained by H & E stain.

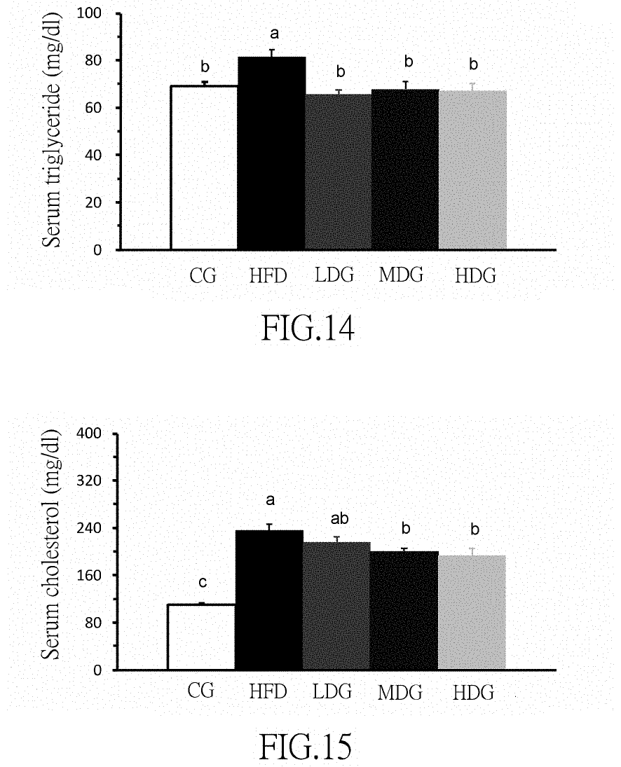

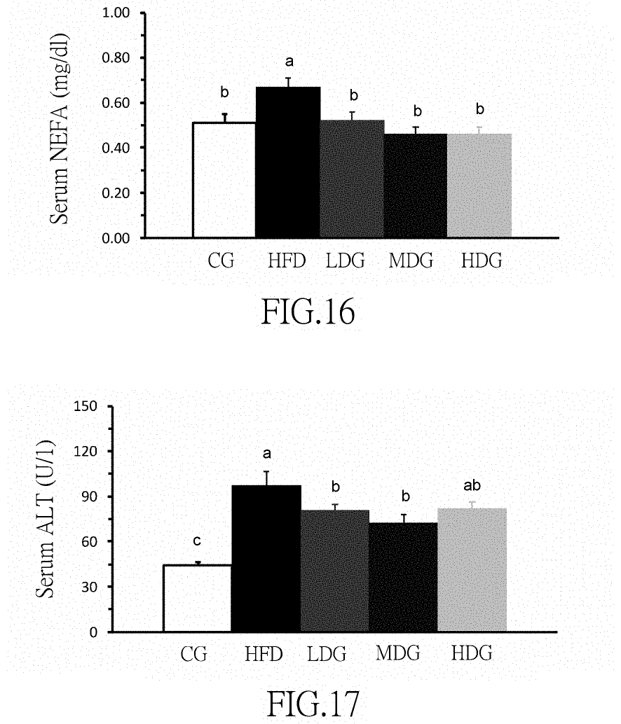

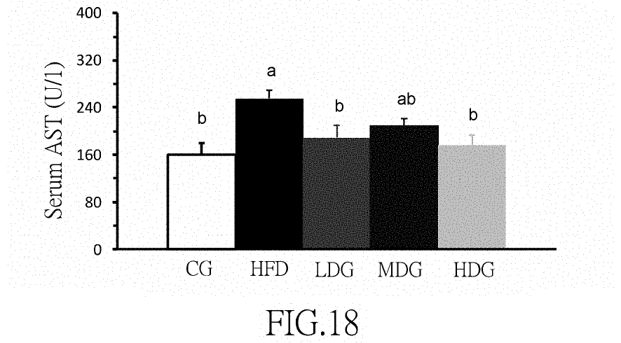

Referring to FIG. 14, serum triglycerides were significantly lower in the low dose group, the medium dose group, and the high dose group compared with the high fat diet group. Referring to FIG. 15, compared with the high fat diet group, the serum cholesterols were dose-dependently decreased in the low dose group, the medium dose group, and the high dose group. Referring to FIG. 16, serum non-esterified fatty acid of the low dose group, the medium dose group, and the high dose group were significantly decreased compared with the high fat diet group. Compared with the high fat diet group, the serum ALT in the low dose group, the medium dose group, and the high dose group were decreased. Referring to FIG. 18, compared with the high fat diet group, the serum AST in the low dose group, the medium dose group, and the high dose group were decreased.

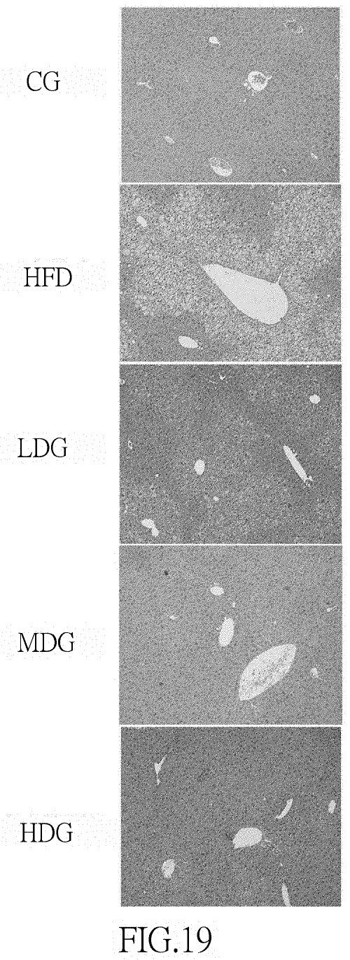

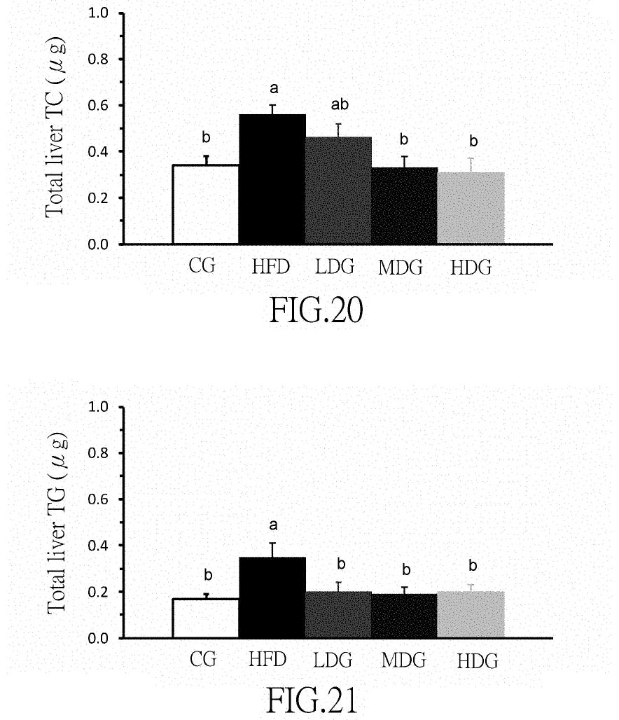

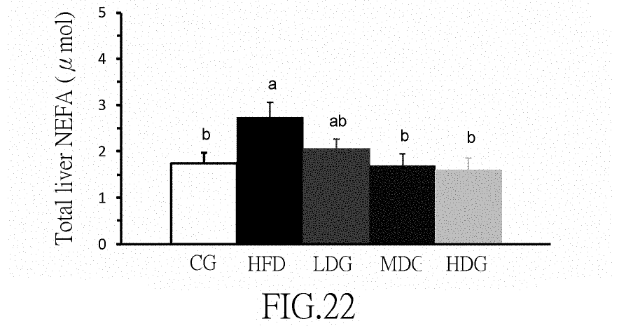

Referring to FIG. 19, the liver tissue of the high fat diet group was filled with fats and appeared hollow and loose. From the low dose group, the medium dose group and the high dose group, the liver cells became denser with the increase of the dose, and the high dose group was similar to the control group. Referring to FIG. 20, compared with the high fat diet group, the total cholesterol of total liver decreased in the low dose group, the medium dose group, and the high dose group. Referring to FIG. 21, the total triglyceride of total liver significantly decreased in the low dose group, the medium dose group, and the high dose group, as compared with the high fat diet group. Referring to FIG. 22, compared with the high fat diet group, the non-esterified fatty acid of total liver decreased in the low dose group, the medium dose group, and the high dose group.

### Example 6: Analysis of Fenugreek Plant Tissue Components

The fenugreek water extract obtained by Preparation Example 1, and a whole-plant fenugreek extract obtained from the fresh or dried whole-plant fenugreek (including the leaves, stem, and roots of the fenugreek) according to the preparation method of Preparation Example 1 were respectively analyzed by HPLC. The analytical conditions were as follows: column: C18 (5 μm×250 mm); mobile phase: methanol:water=90:10; detection wavelength: 203 nm; injection volume: 20 μL; retention time: 30 minutes; and flow rate: 1 mL/min.

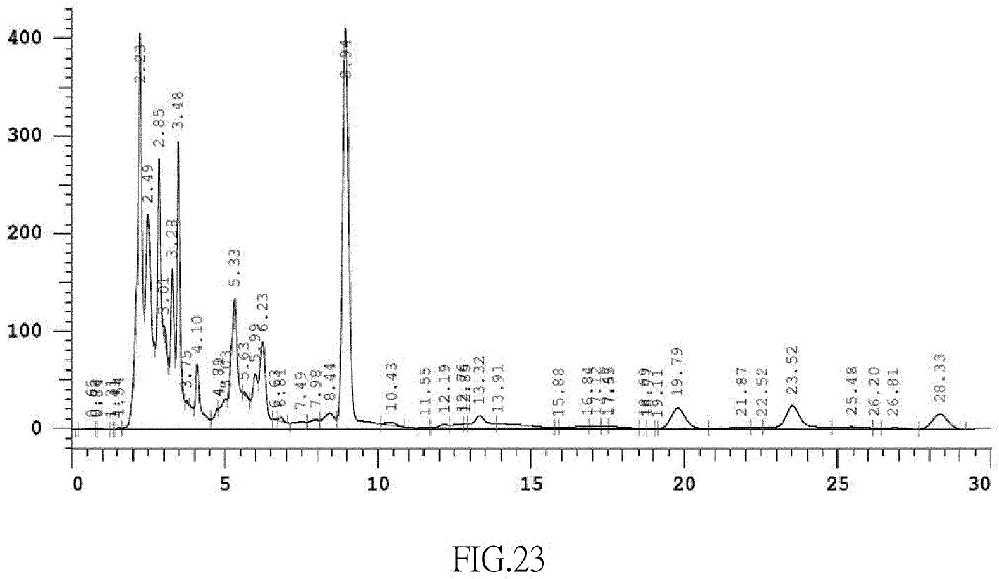

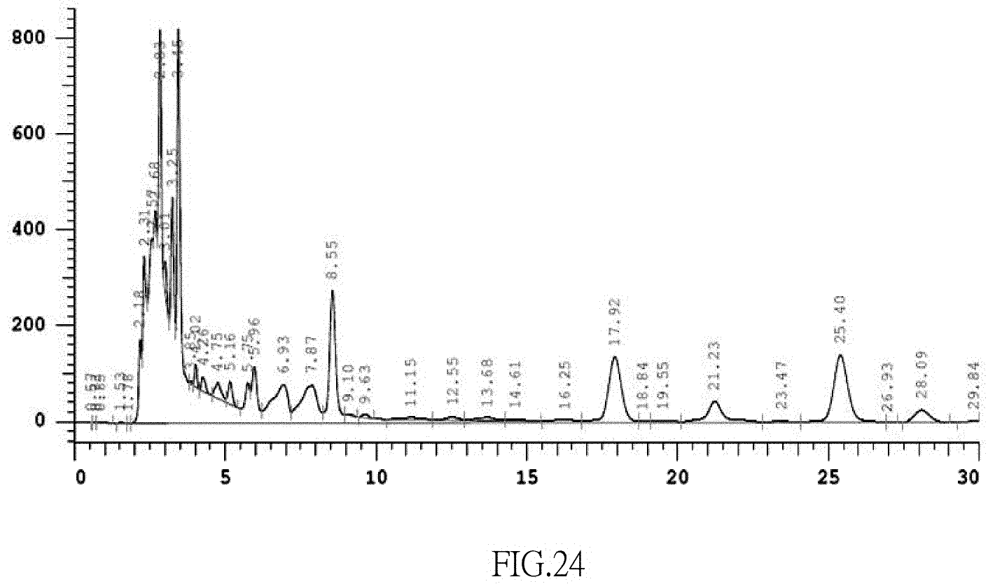

Referring to FIGS. 23 and 24, the results showed that the similarity of the saponins profile of the fenugreek water extract (FIG. 23) and the whole-plant fenugreek extract (FIG. 24) was high, and thus either fenugreek seed or the whole-plant fenugreek extract by the water extract should have a similar effect.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A method for preparing a fenugreek extract, comprising the steps of:
(1) preparing a fenugreek plant tissue, soaking the fenugreek plant tissue in water for 0.5 hour to 5 hours and at a temperature between 25° C. and 100° C. for extraction, to obtain an extracted fenugreek plant tissue; and
(2) filtering the extracted fenugreek plant tissue to obtain the fenugreek extract.

2. The method according to claim 1, wherein the fenugreek plant tissue is fenugreek seed or whole-plant fenugreek.

3. The method according to claim 1, wherein the water is at a weight ratio of 1 to 20 times of the fenugreek plant tissue.

4. The method according to claim 1, wherein the extraction period is between 0.5 hour and 3 hours, and the temperature of the extraction is between 50° C. and 95° C.

5. The method according to claim 2, wherein the extraction period is between 0.5 hour and 3 hours, and the temperature of the extraction is between 50° C. and 95° C.

6. The method according to claim 3, wherein the extraction period is between 0.5 hour and 3 hours, and the temperature of the extraction is between 50° C. and 95° C.

7. A fenugreek extract, which is obtained by the method according to claim 1.

8. An active substance for preventing or treating nonalcoholic fatty liver disease, wherein the active substance comprises the fenugreek extract according to claim 7.

9. The active substance according to claim 8, wherein the active substance comprises red yeast rice extract, rice bran extract, artichoke extract, taurine, ginseng extract or any combination thereof.

10. The active substance according to claim 8, wherein the active substance comprises 12 to 16 parts by weight fenugreek extract, 3 to 14 parts by weight red yeast rice extract, 0.5 to 5 parts by weight rice bran extract, 0.5 to 3 parts by weight artichoke extract, 0.8 to 25 parts by weight taurine, and 0.1 to 10 parts by weight ginseng extract.

11. A pharmaceutical composition for preventing or treating nonalcoholic fatty liver disease, wherein the pharmaceutical composition comprises the active substance according to claim 8 and a pharmaceutically acceptable carrier.
